(19) United States
(12) Patent Application Publication
(10) Pub. No.: US 2020/0383934 A1
(43) Pub. Date: Dec. 10, 2020

(54) CONSTIPATION SPECIFIC TREATMENT FORMULATIONS

(71) Applicant: ColonaryConcepts LLC, Palm Beach Gardens, FL (US)

(72) Inventors: Michael K. ALLIO, Palm Beach Gardens, FL (US); Corey A. SIEGEL, Palm Beach Gardens, FL (US); Joshua KORZENIK, Palm Beach Gardens, FL (US)

(21) Appl. No.: 16/961,905

(22) PCT Filed: Jan. 11, 2019
(86) PCT No.: PCT/US2019/013371

§ 371 Date: Jul. 13, 2020

Related U.S. Application Data
(60) Provisional application No. 62/617,070, filed on Jan. 12, 2018.

(51) Int. Cl. A61K 31/047; A61K 31/716; A61K 35/742; A61K 47/38; A61K 47/14; A61K 31/506; A61K 31/133; A23L 33/10; A23L 33/135; A23L 29/00; A23L 33/12; A23L 33/21

(52) U.S. Cl. CPC A61K 31/047; A61K 31/716; A61K 35/742; A61K 47/38; A61K 47/14; A61K 31/506; A61K 45/06; A23L 33/127; A23L 33/135; A23L 29/035; A23L 33/12; A23L 33/21; A61K 31/133

(57) ABSTRACT

Described herein are compositions and methods that, in some embodiments, are specifically configured to treat different types of constipation. In particular, described herein are compositions and methods for treating IBS-C, opioid induced constipation, and chronic idiopathic constipation. In addition to treating the symptom of constipation, these embodiments of the compositions and methods described herein are configured to address specific etiologies and/or pathophysiologies associated with the constipation types of IBS-C, opioid induced constipation, and chronic idiopathic constipation.

### CROSS-REFERENCE

This application claims the benefit of U.S. Provisional Application No. 62/617,070 filed Jan. 12, 2018 which application is incorporated herein by reference.

### BACKGROUND

Constipation is a widespread condition worldwide. There are a number of different types of constipation as well as different constipation etiologies. Types of constipation include Chronic Idiopathic Constipation (CIC), Irritable Bowel Syndrome with constipation (IBS-C), and drug induced constipation.

### SUMMARY

Different types of constipation exist having different etiologies and/or pathophysiologies and the traditional treatment modalities for constipation do not address these differences in etiology and/or pathophysiology. Rather, the traditional treatment modalities focus solely on treating the symptom of constipation itself while not directly and fully addressing the etiologies and pathophysiologies associated with the condition that causes the constipation, and because the traditional therapies for constipation do not address underlying etiologies and/or pathophysiologies that cause constipation, they do not, generally, effectively treat constipation.

The traditional constipation treatment modalities also do not address multiple symptoms well in patients suffering from constipation. For example, traditional treatment modalities do not address such constipation related symptoms as cramping, bloating, and flatus.

Described herein are compositions and methods for treating constipation, and in particular, the compositions and methods described herein, inter alia, address shortcomings of the traditional therapy for constipation by actively addressing etiologies and/or pathophysiologies that cause constipation.

Described herein are compositions and methods that, in some embodiments, are specifically configured to treat different types of constipation. In particular, described herein are compositions and methods for treating IBS-C, opioid induced constipation (OIC), and CIC. In addition to treating the symptom of constipation, these embodiments of the compositions and methods described herein are configured to address specific etiologies and/or pathophysiologies associated with the constipation types of IBS-C, OIC, and CIC.

### IBS-C

IBS-C is a condition associated with the symptoms of abdominal pain, flatus, bloating, and constipation. A significant factor in the etiology of the disease is a nervous system abnormality that affects nerves in the gastro-intestinal (GI) tract resulting in dysmotility and overall decreased brain-GI coordination. The nervous system component of the disease is either caused by or at least highly affected by psychiatric conditions including, for example, anxiety and depression. As such, the type-specific formulations described herein include treatments of the etiology of the IBS-C syndrome—nervous system disorder(s)—in order to more directly address the pathophysiology of the syndrome—dysmotility and overall decreased brain-GI coordination. Namely, in embodiments described herein, a composition for treating IBS-C comprises an anxiolytic and/or an anti-depressant that is directed to treating an associated condition that causes or affects constipation in IBS-C.

Described herein is a composition for treating constipation comprising: a laxative; and an anxiolytic. In some embodiments, the laxative comprises PEG. In some embodiments, the laxative comprises PEG 3350. In some embodiments, the anxiolytic is a medication that increases serotonin within a body of an individual. In some embodiments, the anxiolytic comprises Buspirone. In some embodiments, the anxiolytic comprises a tricyclic anti-depressant. In some embodiments, the anxiolytic comprises a selective serotonin reuptake inhibitor. In some embodiments, the selective serotonin reuptake inhibitor comprises Prozac. In some embodiments, the anxiolytic comprises Propranolol. In some embodiments, the anxiolytic comprises a serotonin precursor. In some embodiments, the serotonin precursor is one of L-tryptophan, L-phenylalanine, and L-tyrosine. In some embodiments, the composition comprises one or more food ingredients, wherein the one or more food ingredients are mixed together with the laxative and the anxiolytic forming a solid food item containing the laxative and the anxiolytic. In some embodiments, the composition comprises at least 2 g of fiber. In some embodiments, the composition comprises a probiotic. In some embodiments, the composition comprises a prebiotic. In some embodiments, the composition comprises butyrate.

### OIC

OIC is associated with partial or complete GI dysmotility (and in severe cases ileus) causing severe constipation and associated symptoms resulting from chronic intake or a single intake of a large dose of an opioid based medication which interacts with GI cell receptors to cause dysmotility. Individuals taking opioids often do so in order to treat severe and/or chronic pain and as such are typically unable to forgo taking the opioids despite the constipation symptoms. As such, the type-specific formulations described herein include treatments of the pathophysiology of OIC—GI stasis—as well as the etiology of OIC—the opioid interaction with GI cell receptors. Namely, in embodiments described herein, a composition for treating OIC comprises one or more stimulant laxatives and one or more motility promoting agents, and, in some embodiments of the formulations described herein, the formulations include GI specific cell receptor blockers that interfere with opioid-receptor binding within the GI tract. In this way, the GI stasis is addressed by a stimulant laxative and/or a motility promoting agent and the opioids effect on GI cells is blocked by the opioid receptor blocking agent.

Described herein is a composition for treating constipation comprising: a laxative; and a motility promoting agent. In some embodiments, the laxative comprises PEG. In some embodiments, the laxative comprises PEG 3350. In some embodiments, the laxative comprises a stimulant laxative. In some embodiments, the stimulant laxative comprises bisacodyl. In some embodiments, the motility promoting agent comprises neostigmine. In some embodiments, the motility promoting agent comprises fiber. In some embodiments, the composition comprises one or more food ingredients, wherein the one or more food ingredients are mixed together with the laxative and the motility promoting agent forming a solid food item containing the laxative and the motility promoting agent. In some embodiments, the composition comprises a probiotic. In some embodiments, the composition comprises a prebiotic. In some embodiments, the composition comprises butyrate.

### CIC

CIC is the presence of a relatively long-standing constipation in an individual typically not associated with additional GI symptoms such as bloating and abdominal pain. CIC is associated with poor or diminished overall GI function and health. As such, the type-specific formulations described herein include treatments that promote overall GI health. Namely, in embodiments described herein, a composition for treating CIC includes one or more of a prebiotic or a probiotic and/or an additional substance that promotes GI health.

Described herein is a composition for treating constipation comprising: a laxative; and one or more of a probiotic and a prebiotic. In some embodiments, the laxative comprises PEG. In some embodiments, the laxative comprises PEG 3350. In some embodiments, the probiotic is one of Bacillus subtilis, Bacillus clausii, Bacillus cereus, Bacillus coagulans and Bacillus licheniformis. In some embodiments, the prebiotic comprises beta-glucan. In some embodiments, the composition comprises one or more food ingredients, wherein the one or more food ingredients are mixed together with the laxative and the one or more of the probiotic and the prebiotic. In some embodiments, the composition comprises at least 2 g of fiber. In some embodiments, the composition comprises butyrate.

### DETAILED DESCRIPTION

Described herein are compositions and methods configured for treating constipation including compositions and formulations directed to treating specific types of constipation including IBS-C, OIC, and CIC. More specifically, certain embodiments of the compositions and methods described herein are configured to address an etiology and/or a pathophysiology associated with a specific type of constipation. In addition, the compositions described herein are not limited to the treatment of these specific types of constipation only but rather are beneficial in the treatment of all types of constipation.

In general, the compositions described herein comprise formulations that combine a laxative with one or more additional active agents that target (i.e. treat) a specific etiology and/or pathophysiology associated with a type of constipation. In some embodiments of the compositions described herein comprise one or more laxatives. Non-limiting examples of laxatives suitable for combination with the one or more food ingredients include dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG) (including, for example, PEG 3350, PEG 4000, PEG 6000, and PEG 8000), vitamin C, dioctyl sulfosuccinate (Docusate), bisacodyl (Dulcolax), castor oil, lactitol, and sorbitol.

In some formulations an additional active agent is configured to treat or prevent a symptom associated with constipation such as gas, bloating, abdominal pain, and flatus. Non-limiting examples of active agents used to treat or prevent symptoms associated with constipation include simethicone, calcium carbonate, and ibuprofen.

Formulations of certain embodiments of the compositions described herein include one or more antibiotics. Non-limiting examples of antibiotics used herein include rifaximin and augmentin.

Formulations of certain embodiments of the compositions described herein include one or more food ingredients. In some embodiments of these formulations, the one or more food ingredients are mixed together with one or more active agents forming a food item or a drink. Non-limiting examples of solid food items include food bars, baked goods, meat products, fruit and fruit products, vegetables and plant based products, candies, gums, nuts and nut products. Non-limiting examples of liquid food items include shakes, sodas, milks, coffees, teas, and flavored water based drinks.

In some embodiments, the food item comprises food ingredients that may comprise any edible food ingredients used to make a food item. Non-limiting examples of ingredients include sugars and natural sweeteners, artificial sweeteners, grain based flour, non-grain based flour, fruit and fruit products, vegetables and vegetable products, dairy products including milk, cream, cheese, and butter, edible oils, chocolate, and nuts.

In some embodiments, one or more ingredients comply with the standards of the National Formulary of the U.S. Pharmacopeial Convention (USP/NF). In some embodiments, a USP/NF grade food ingredient comprises a sweetener. Non-limiting examples of USP/NF grade sweeteners include syrup, invert syrup, erythritol, maltodextrin, and dextrin. In some embodiments, a USP/NF grade ingredient comprises a texturizer. Other non-limiting examples USP/NF grade food ingredients suitable for use with the compositions and methods described herein include cocoa butter, malic acid, citric acid, lemon oil, and vanilla flavor. In some embodiments, the laxative comprises a USP/NF grade laxative. Compositions described herein containing USP/NF grade foods and laxatives are beneficial in that, for example, such compositions provide for improved mouthfeel. The use of USP/NF food ingredients in the compositions described herein is also beneficial in that, for example, it provides the ability to include higher amounts of laxative in said compositions than would be possible if non-USP/NF ingredients were used.

In some embodiments, a compound for treating constipation further comprises a binder, wherein a binder is an element used to bind one or more elements of the compound together. In some embodiments, the binder is edible. In some embodiments, the binder comprises cocoa butter. In some embodiments, the binder comprises coconut oil. In some embodiments, the binder comprises cocoa butter. In some embodiments, the binder comprises sunflower seed oil. In some embodiments, a binder is heated and the elements of the compound are added to the heated binder. In some embodiments, one or more elements of the compound are added to the binder at the hottest temperature to which it is heated. In some embodiments, one or more elements of the compound are added to the heated binder after it has cooled. In some embodiments, the binder is a liquid when heated so that when mixed with the elements of the compound, the elements coalesce or are “bound” together by the binder. In an exemplary embodiment, a binder in the compound is in a solid state at room temperature and a liquid state at a relatively small increase in temperature above room temperature. This exemplary binder is used in binding the elements of the compound when a solid compound is used such as, for example, a bar. In the liquid state the binder can coalesce the elements, and, as stated, the exemplary binder is in a liquid state at temperatures slightly above room temperature, thus the other elements of the compound are not heated due to the binder to a large extent which is especially important with respect to the laxatives that are damaged at high temperatures.

The compositions and methods described herein, in some embodiments, comprise fiber. In some embodiments, a composition as described herein comprises 15 grams of fiber. In some embodiments, a composition as described herein comprises 14 grams of fiber. In some embodiments, a composition as described herein comprises 13 grams of fiber. In some embodiments, a composition as described herein comprises 12 grams of fiber. In some embodiments, a composition as described herein comprises 11 grams of fiber. In some embodiments, a composition as described herein comprises 10 grams of fiber. In some embodiments, a composition as described herein comprises 9 grams of fiber. In some embodiments, a composition as described herein comprises 8 grams of fiber. In some embodiments, a composition as described herein comprises 7 grams of fiber. In some embodiments, a composition as described herein comprises 6 grams of fiber. In some embodiments, a composition as described herein comprises 5 grams of fiber. For example, in some embodiments, the composition comprises 4 g of fiber. For example, in some embodiments, the composition comprises 3 g of fiber. For example, in some embodiments, the composition comprises 2 g of fiber. For example, in some embodiments, the composition comprises 1 g of fiber.

Fiber incorporated into some of the embodiments described herein is 100% water soluble. In some embodiments, fiber as used herein is 95% water soluble. In some embodiments, fiber as used herein is 90% water soluble. In some embodiments, fiber as used herein is 85% water soluble. In some embodiments, fiber as used herein is 80% water soluble. In some embodiments, fiber as used herein is 75% water soluble. In some embodiments, fiber as used herein is 70% water soluble. In some embodiments, fiber as used herein is 65% water soluble. In some embodiments, fiber as used herein is 65% water soluble. In some embodiments, fiber as used herein is 60% water soluble. In some embodiments, fiber as used herein is 55% water soluble. In some embodiments, fiber as used herein is 50% water soluble.

Non-limiting examples of suitable fiber sources include Methylcellulose, Calcium Polycarbophil, Psyllium Husk, and Inulin. In some embodiments, a composition as described herein comprises 2 g of Methylcellulose. In some embodiments, a composition as described herein comprises 1 g of Calcium Polycarbophil. In some embodiments, a composition as described herein comprises 3.4 g of Psyllium Husk. In some embodiments, a composition as described herein comprises 3 g of Inulin.

The compositions described herein, in some embodiments, comprise formulations in the form of a tablet, capsule, wafer, or other type of pill. In some embodiments, the compositions described herein comprise liquid formulations. It should be understood that these pill and liquid formulations, in some embodiments include additional excipients. It should be further understood that the pill formulations as described herein, in some embodiments, are coated or encapsulated.

### IBS-C

Described herein are compositions and methods for treating constipation caused by IBS-C. In addition to treating constipation, embodiments of the compositions described herein are configured to treat a neurological disorder that causes and/or is associated with the constipation symptoms in IBS-C. In these embodiments, a composition for treating constipation in IBS-C comprises a laxative combined with one of or both of an anxiolytic and an antidepressant. That is, in some embodiments, the compositions as described herein comprise formulations that combine a laxative with one or more of an anxiolytic and an antidepressant in a single formulation in the form of, for example, a tablet, capsule, wafer (or other type of pill) or are combined within a food item.

In addition, in certain embodiments, modular dosing is achieved by providing a first formulation comprising, for example, an anxiolytic and/or an antidepressant and a second formulation comprising, for example, a laxative wherein the two formulations are to be taken by an individual concurrently.

Non-limiting examples of anxiolytics suitable for use with the compositions described herein include Alprazolam (Alprazolam (Xanax)), Bromazepam (Lectopam, Lexotan), Buspar (Buspirone), Chlordiazepoxide (Librium), Clonazepam (Klonopin, Rivotril), Clorazepate (Tranxene), Diazepam (Valium), Flurazepam (Dalmane), Lorazepam (Ativan), Oxazepam (Serax, Serapax), Temazepam (Restoril), and Triazolam (Halcion).

Alprazolam, for example, is provided at or between 0.25 mg to 5 mg in various embodiments of the compositions described herein. Bromazepam, for example, is provided at or between 6 mg to 18 mg in various embodiments of the compositions described herein. Buspar, for example, is provided at or between 7.5 mg to 30 mg in various embodiments of the compositions described herein. Chlordiazepoxide, for example, is provided at or between 5 mg to 25 mg in various embodiments of the compositions described herein. Clonazepam, for example, is provided at or between 0.125 mg to 2 mg in various embodiments of the compositions described herein. Clorazepate, for example, is provided at or between 3.755 mg to 15 mg in various embodiments of the compositions described herein. Diazepam, for example, is provided at or between 2 mg to 10 mg in various embodiments of the compositions described herein. Flurazepam, for example, is provided at or between 15mg to 30 mg in various embodiments of the compositions described herein. Lorazepam, for example, is provided at or between 0.5 mg to 2 mg in various embodiments of the compositions described herein. Oxazepam, for example, is provided at or between 10 mg to 30 mg in various embodiments of the compositions described herein. Temazepam, for example, is provided at or between 7.5 mg to 30 mg in various embodiments of the compositions described herein. Triazolam, for example, is provided at or between 0.125 mg to 25 mg in various embodiments of the compositions described herein. As stated, the doses provided here are examples of doses used in various embodiments and are not intended to be limiting as other doses are effective as well in certain patient populations.

Non-limiting examples of anti-depressants suitable for use with the compositions described herein include the classes of serotonin reuptake inhibitors and tricyclic anti-dipressents such as Amitriptyline (Elavil, Endep), Amitriptylinoxide (Amioxid, Ambivalon, Equilibrin), Clomipramine (Anafranil), Desipramine (Norpramin, Pertofrane), Dibenzepin (Noveril, Victoril), Dimetacrine (Istonil), Dosulepin (Prothiaden), Doxepin (Adapin, Sinequan), Imipramine (Tofranil), Lofepramine (Lomont, Gamanil), Melitracen (Dixeran, Melixeran, Trausabun), Nitroxazepine (Sintamil), Nortriptyline (Pamelor, Aventyl), Noxiptiline (Agedal, Elronon, Nogedal), Opipramol (Insidon), Pipofezine (Azafen/Azaphen), Protriptyline (Vivactil), Trimipramine (Surmontil), Amitriptyline (Elavil, Endep), Amitriptylinoxide (Amioxid, Ambivalon, Equilibrin, Clomipramine (Anafranil), Desipramine (Norpramin, Pertofrane), Dibenzepin (Noveril, Victoril), Dimetacrine (Istonil), Dosulepin (Prothiaden), Doxepin (Adapin, Sinequan), Imipramine (Tofranil), Lofepramine (Lomont, Gamanil), Melitracen (Dixeran, Melixeran, Trausabun), Nitroxazepine (Sintamil), Nortriptyline (Pamelor, Aventyl), Noxiptiline (Agedal, Elronon, Nogedal), Opipramol (Insidon), Pipofezine (Azafen/Azaphen), Protriptyline (Vivactil), and Trimipramine (Surmontil).

In some embodiments of the compositions and methods described herein, the compositions comprise a serotonin precursor. Non-limiting examples of serotonin precursors include L-tryptophan, L-phenylalanine, and L-tyrosine.

An exemplary embodiment of a composition as described herein for treating constipation caused by IBS-C comprises PEG 3350 and Buspirone in tablet form. In a second exemplary embodiment, a composition for treating constipation caused by IBS-C comprises PEG 3350 and Alprazolam (Xanax) mixed together with one or more food ingredients to form a food item containing PEG 3350 and Alprazolam (Xanax). In a third exemplary embodiment, a composition for treating constipation caused by IBS-C comprises Lactitol and Buspirone in capsule form. In a fourth exemplary embodiment, a composition caused by IBS-C comprises Lactitol and Fluoxetine (Prozac) mixed together with one or more food ingredients to form a food item. In a fifth exemplary embodiment, a composition for treating constipation caused by IBS-C comprises PEG 3350 and Lactitol and either Alprazolam (Xanax) or Fluoxetine (Prozac). In a sixth embodiment, a composition for treating constipation caused by IBS-C comprises lactilol, Fluoxetine (Prozac), and L-tryptophan. It should be understood that many combinations of laxative, anxiolytic and/or antidepressant, and/or serotonin precursor are suitable for use in the compositions and methods described herein in different formulations and further combinable in numerous different ways using modular dosing.

In some embodiments of compositions described herein a laxative is provided together with a probiotic and/or a prebiotic and/or an additional substance that promotes GI health. That is, in some embodiments, the compositions as described herein comprise formulations that combine a laxative with one or more of a probiotic, a prebiotic and an additional gastro-intestinal health promoting agent in a single formulation in the form of, for example, a tablet, capsule, wafer (or other type of pill) or within a food item. In addition, in certain embodiments, modular dosing is achieved by providing, for example, a first formulation comprising a laxative and/or probiotic and/or an prebiotic and a second formulation comprising a laxative and/or an additional GI health promoting agent wherein the two formulations are to be taken by an individual concurrently.

Non-limiting examples of laxatives suitable for use in the compositions and methods configured to treat IBS-C include dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG) (including, for example, PEG 3350, PEG 4000, PEG 6000, and PEG 8000), vitamin C, dioctyl sulfosuccinate (Docusate), bisacodyl (Dulcolax), castor oil, lactitol, and sorbitol.

Non-limiting examples of probiotics suitable for use in the compositions and methods configured to treat IBS-C include strains from the following genera:

Lactobacillus, Bifidobacterium, Saccharomyces, Enterococcus, Streptococcus, Pediococcus, Leuconostoc, Bacillus, Escherichia coli. Non-limiting examples of specific micro-organisms suitable for use in the compositions described herein include Lactobacillus acidophilus, L. casei, L. paracasei, L. rhamnosus, L. delbrueckii subsp. bulgaricus, L. brevis, L. johnsonii, L. plantarum, L. fermentum, Bifidobacterium infantis, B. adolescentis, B. animalis subsp animalis, B. animalis subsp lactis, B. bifidum, B. longum, B. breve, B. infantis, Saccharomyces bayanus, S. cervisiae, S. boulardii. Lactococcus lactis subsp. Lactis, Streptococcus pyogenes, Streptococcus pneumoniae, S. thermophiles, vancomycin-resistant Enterococcus faecium, B. subtilis, B. coagulans, B. subtilis, B. cereus, B. indicus, B. licheniformis, and Escherichia coli. VSL#3 is an example of a probiotic product suitable for use with the compositions described herein and ranges in dose from 100 Billion to 900 Billion CFUs (colony forming unit).

Non-limiting examples of prebiotics suitable for use in the compositions and methods configured to treat CIC include fructo-oligosaccharide, galacto-oligosaccharide, lactulose, beta-glucan, inulin, pectin and digestion resistant starch.

An exemplary substance that promotes GI health is butyrate, which is also produced by certain probiotics. In some embodiments, a composition as described herein for treating IBS-C includes butyrate. Butyrate is beneficial in that, for example, it reduces abdominal pain in IBS-C sufferers. In some embodiments, butyrate is microencapsulated within the formulation.

### OIC

Described herein are compositions and methods for treating OIC. The compositions and methods described herein in particular are directed to addressing, inter alia, gastrointestinal stasis caused by opioids. For example, embodiments of compositions described herein comprise a laxative which in some of these embodiments further comprises a stimulant laxative. In addition, certain embodiments of the compositions described herein comprise a motility promoting agent. Both a laxatives, and especially stimulant laxatives, and motility promoting agents are configured to counteract GI stasis.

In addition, certain embodiments of the compositions described herein comprise an opioid receptor blocking agent. An opioid receptor blocking agent, as described herein, is configured to specifically affect and exclusively (or nearly exclusively) target opioid receptors of the GI tract. That is, in some embodiments, an opioid receptor blocker as used herein is combined with one or more antibodies configured to target and specifically bind with a GI opioid receptor. In some embodiments, the form of delivery of the opioid receptor blocking agent prevents its being absorbed (through the GI tract) into the blood stream. One way this is achieved, in some embodiments, is through binding the opioid receptor blocking agent to a molecule that will not cross the GI-blood barrier and as such the opioid receptor blocking agent will only have localized effect within the GI tract.

In some embodiments of the compositions described herein, a composition for treating OIC comprises a laxative and a motility promoting agent. In some embodiments of the compositions described herein, a composition for treating OIC comprises a laxative and an opioid receptor blocking agent. In some embodiments of the compositions described herein, a composition for treating OIC comprises a laxative, a motility stimulating agent, and an opioid receptor blocking agent. That is, in some embodiments, the compositions as described herein comprise formulations that combine a laxative with one or more of a motility promoting agent and opioid receptor blocking agent in a single formulation in the form of, for example, a tablet, capsule, wafer, or other type of pill or within a food item. In addition, in certain embodiments, modular dosing is achieved by providing, for example, a first formulation comprising a laxative and a motility promoting agent and a second formulation comprising a an opioid receptor blocker and/or a second laxative and/or a second motility promoting agent wherein the two formulations are to be taken by an individual concurrently.

Non-limiting examples, of stimulant laxatives suitable for use with compositions and methods configured to treat OIC include Senna and Bisacodyl (Dulcolax). Non limiting examples of other laxatives suitable for use in the compositions and methods described herein for treating OIC include dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG) (including, for example, PEG 3350, PEG 4000, PEG 6000, and PEG 8000), vitamin C, dioctyl sulfosuccinate (Docusate), castor oil, lactitol, and sorbitol.

Non-limiting examples of motility promoting agents suitable for use with compounds and methods configured to treat OIC include Fiber, Neostigmine, Tegaserod (Zelnorm), and Prucalopride (Resolor).

Non-limiting examples of opioid receptor blockers suitable for use with compounds and methods configured to treat OIC include Naloxone, Naltrexone, methylnaltrexone (Relistor), and Nalmefene.

An exemplary embodiment of a composition for treating OIC comprises Bisacodyl and Tegaserod in tablet form. In a second exemplary embodiment, a composition for treating OIC comprises Senna and Neostigmine mixed together with one or more food ingredients to form a food item containing Senna and Neostigmine. In a third exemplary embodiment, a composition for treating OIC comprises Bisacodyl and Naloxone in capsule form. In a fourth exemplary embodiment, a composition for treating OIC comprises Senna and Prucalopride mixed together with one or more food ingredients to form a food item. In a fifth exemplary embodiment, a composition for treating OIC comprises Bisacodyl and Senna and either Prucalopride or Naltrexone. In a sixth embodiment, a composition for treating OIC comprises Bisacodyl, Neostigmine, and Nalmefene. It should be understood that many combinations of laxative, motility promoting agent and/or opioid receptor blocker are suitable for use in the compositions and methods described herein in different forms.

### CIC

Described herein are compositions and methods for treating CIC. The compositions and methods described herein in particular are directed to addressing, inter alia, gastrointestinal health. For example, embodiments of compositions described herein comprise a laxative together with a probiotic and/or a prebiotic and/or an additional substance that promotes GI health. That is, in some embodiments, the compositions as described herein comprise formulations that combine a laxative with one or more of a probiotic, a prebiotic and an additional gastro-intestinal health promoting agent in a single formulation in the form of, for example, a tablet, capsule, wafer (or other type of pill) or within a food item. In addition, in certain embodiments, modular dosing is achieved by providing, for example, a first formulation comprising a laxative and/or probiotic and/or an prebiotic and a second formulation comprising a laxative and/or an additional GI health promoting agent wherein the two formulations are to be taken by an individual concurrently.

Non-limiting examples of laxatives suitable for use in the compositions and methods configured to treat CIC include dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG) (including, for example, PEG 3350, PEG 4000, PEG 6000, and PEG 8000), vitamin C, dioctyl sulfosuccinate (Docusate), bisacodyl (Dulcolax), castor oil, lactitol, and sorbitol.

Non-limiting examples of probiotics suitable for use in the compositions and methods configured to treat CIC include strains from the following genera:

Lactobacillus, Bifidobacterium, Saccharomyces, Enterococcus, Streptococcus, Pediococcus, Leuconostoc, Bacillus, Escherichia coli. Non-limiting examples of specific micro-organisms suitable for use in the compositions described herein include Lactobacillus acidophilus, L. casei, L. paracasei, L. rhamnosus, L. delbrueckii subsp. bulgaricus, L. brevis, L. johnsonii, L. plantarum, L. fermentum, Bifidobacterium infantis, B. adolescentis, B. animalis subsp animalis, B. animalis subsp lactis, B. bifidum, B. longum, B. breve, B. infantis, Saccharomyces bayanus, S. cervisiae, S. boulardii. Lactococcus lactis subsp. Lactis, Streptococcus pyogenes, Streptococcus pneumoniae, S. thermophiles, vancomycin-resistant Enterococcus faecium, B. subtilis, B. coagulans, B. subtilis, B. cereus, B. indicus, B. licheniformis, and Escherichia coli. VSL#3 is an example of a probiotic product suitable for use with the compositions described herein and ranges in dose from 100 Billion to 900 Billion CFUs (colony forming unit).

Non-limiting examples of prebiotics suitable for use in the compositions and methods configured to treat CIC include fructo-oligosaccharide, galacto-oligosaccharide, lactulose, beta-glucan, inulin, pectin and digestion resistant starch.

An exemplary substance that promotes GI health is butyrate, which is also produced by certain probiotics. In some embodiments, butyrate is microencapsulated within the formulation.

An exemplary embodiment of a composition for treating CIC comprises PEG 3350 and butyrate in tablet form. In a second exemplary embodiment, a composition for treating CIC comprises Senna and a probiotic comprising B. subtilits mixed together with one or more food ingredients to form a food item containing Senna and B. subtilis. In a third exemplary embodiment, a composition for treating CIC comprises lactitol, B. subtilits, and Beta-Glucan in capsule form. In a fourth exemplary embodiment, a composition for treating CIC comprises PEG 3350 and B. subtilits mixed together with one or more food ingredients to form a food item. In a fifth exemplary embodiment, a composition for treating CIC comprises lactitol and either PEG 3350 or B. subtilits. In a sixth embodiment, a composition for treating CIC comprises PEG 3350, B. subtilits, and B. breve, and/or butyrate. It should be understood that many combinations of laxative, probiotic, prebiotic, and other substance for promoting GI health are suitable for use in the compositions and methods described herein in different forms.

### Methods of Treatment

Described herein are methods for treating specific types of constipation including IBS-C, OIC, and CIC. In some embodiments, an individual ingests a composition as described herein comprising at least one active agent configured to address either an etiology or a pathophysiology of a specific type of constipation. In some embodiments, an individual first ingests a first composition and concurrently ingests a second composition. In this embodiment, the first composition and the second composition comprise different active agents and/or comprise different formulations wherein the first and the second compositions in combination are configured to treat an etiology and/or a pathophysiology of a specific type of constipation.

As noted, the composition and methods as described herein are suitable for use in the specific types of constipation of IBS-C, OIC, and CIC, and in addition, the teachings herein are also relevant to other types of constipation. In some embodiments, the constipation treated by the compositions and methods described herein is caused by a disease process such as an anxiety disorder. In some embodiments, the constipation treated by the compositions and methods described herein is caused by aging. In some embodiments, the constipation treated by the compositions and methods described herein is caused by a wasting disease such as certain cancers. In some embodiments, the constipation treated by the compositions and methods described herein is caused by poor nutrition. In some embodiments, the constipation treated by the compositions and methods described herein is caused by a medication. In some embodiments, the constipation treated by the compositions and methods described herein is caused by an opioid. In some embodiments, the constipation treated by the compositions and methods described herein is caused by codeine. In some embodiments, the constipation treated by the compositions and methods described herein is caused by oxycodone. In some embodiments, the constipation treated by the compositions and methods described herein is caused by hydromorphone. In some embodiments, the constipation treated by the compositions and methods described herein is caused by buprenoprhine. In some embodiments, the constipation treated by the compositions and methods described herein is caused by fentanyl. In some embodiments, the constipation treated by the compositions and methods described herein is caused by hydrocodone. In some embodiments, the constipation treated by the compositions and methods described herein is caused by meperidine. In some embodiments, the constipation treated by the compositions and methods described herein is caused by methadone. In some embodiments, the constipation treated by the compositions and methods described herein is caused by morphine sulfate. In some embodiments, the constipation treated by the compositions and methods described herein is caused by oxymorphone. In some embodiments, the constipation treated by the compositions and methods described herein is caused by tramadol. In some embodiments, the constipation treated by the compositions and methods described herein is caused by an antidepressant. In some embodiments, the constipation treated by the compositions and methods described herein is caused by amitriptyline. In some embodiments, the constipation treated by the compositions and methods described herein is caused by imipramine. In some embodiments, the constipation treated by the compositions and methods described herein is caused by doxepin. In some embodiments, the constipation treated by the compositions and methods described herein is caused by an antihypertensive. In some embodiments, the constipation treated by the compositions and methods described herein is caused by clonidine. In some embodiments, the constipation treated by the compositions and methods described herein is caused by a beta blocker. In some embodiments, the constipation treated by the compositions and methods described herein is caused by atenolol. In some embodiments, the constipation treated by the compositions and methods described herein is caused by an anti-Parkinson's agent. In some embodiments, the constipation treated by the compositions and methods described herein is caused by bromocriptine. In some embodiments, the constipation treated by the compositions and methods described herein is caused by a cholesterol lowering agent. In some embodiments, the constipation treated by the compositions and methods described herein is caused by cholestyramine. In some embodiments, the constipation treated by the compositions and methods described herein is caused by a medication for treatment of gastrointestinal ulcers. In some embodiments, the constipation treated by the compositions and methods described herein is caused by sucralfate. In some embodiments, the constipation treated by the compositions and methods described herein is caused by an anticonvulsant. In some embodiments, the constipation treated by the compositions and methods described herein is caused by phenytoin. In some embodiments, the constipation treated by the compositions and methods described herein is caused by carbamazapine. In some embodiments, the constipation treated by the compositions and methods described herein is caused by an iron supplement. In some embodiments, the constipation treated by the compositions and methods described herein is caused by a calcium channel blocker. In some embodiments, the constipation treated by the compositions and methods described herein is caused by diltiazem. In some embodiments, the constipation treated by the compositions and methods described herein is caused by nifedipine. In some embodiments, the constipation treated by the compositions and methods described herein is caused by an NSAID. In some embodiments, the constipation treated by the compositions and methods described herein is caused by ibuprofen. In some embodiments, the constipation treated by the compositions and methods described herein is caused by an anticholinergic. In some embodiments, the constipation treated by the compositions and methods described herein is caused by diphenhydramine. In some embodiments, the constipation treated by the compositions and methods described herein is caused by cetirizine. In some embodiments, the constipation treated by the compositions and methods described herein is caused by fexofenadine.

### Manufacture

Any of the embodiments of the compositions described herein are prepared utilizing a low temperature manufacturing process, so that, in particular, minimal (or no) heat is applied to one or more laxatives within the compositions described herein. It should be understood, that the application of high temperature to a laxative or other medicament will cause degradation of the laxative or medicament decreasing the efficacy of the laxative or medicament as well as potentially releasing toxic degradation products. In the instant described manufacturing methods, the efficacy of the laxative or other medicament is maintained and the release of potentially toxic degradation products is avoided.

An exemplary method of making a composition as described herein comprises (1) mixing the active ingredient (e.g. laxative and/or anxiolytic) with dry ingredients, for example, sea salt or flavor powders; (2) blending the active ingredient mixture with a melted binder and/or glycerin, preferably at room temperature, for example, a temperature around or below 80° F. (26.7° C.), to form a homogenous malleable semi-solid mixture (for example, a fondant-like mass); (3) blending the malleable semi-solid mixture with rice syrup to form a homogeneous dispersion; (4) optionally folding additional ingredients, for example, rice crisps, dried fruit, or chocolate chips, into the dispersion; and (5) forming the dispersion into a bar, for example, of about 35 or 70 grams.

Non-limiting examples of processes for manufacturing a composition comprising a solid food item as described herein include mixing, cooking, and baking one or more food ingredients and/or one or more laxatives. In some embodiments, the one or more food ingredients and the one or more laxatives all coalesce together. When one or more food ingredients and/or one or more laxatives are heated in order to manufacture the food item, the heating is done at a temperature wherein the food ingredients tend to coalesce together (i.e. with each other as well as with one or more laxatives) while the laxatives do not degrade. In addition, typically, heat is not applied at or above a temperature at which a therapeutic property of one or more laxatives of the compound are affected. The effect of heating is determined by both the temperature and duration over which heat is applied. In some embodiments, a compound for treating constipation comprises a mixture of one or more laxatives and one or more food ingredients which are heated to a temperature at or above 350 degrees Fahrenheit. In some embodiments, a compound for treating constipation comprises a mixture of one or more laxatives and one or more food ingredients which are heated to a temperature at or above 300 degrees Fahrenheit. In some embodiments, a compound for treating constipation comprises a mixture of one or more laxatives and one or more food ingredients which are heated to a temperature at or above 250 degrees Fahrenheit. In some embodiments, a compound for treating constipation comprises a mixture of one or more laxatives and one or more food ingredients which are heated to a temperature at or above 200 degrees Fahrenheit. In some embodiments, a compound for treating constipation comprises a mixture of one or more laxatives and one or more food ingredients which are heated to a temperature at or above 150 degrees Fahrenheit. In some embodiments, a compound for treating constipation comprises a mixture of one or more laxatives and one or more food ingredients which are heated to a temperature at or above 100 degrees Fahrenheit. In some embodiments, a compound for treating constipation comprises a mixture of one or more laxatives and one or more food ingredients which are heated to a temperature at or above 95 degrees Fahrenheit. In some embodiments, a compound for treating constipation comprises a mixture of one or more laxatives and one or more food ingredients which are heated to a temperature at or above 90 degrees Fahrenheit. In some embodiments, a compound for treating constipation comprises a mixture of one or more laxatives and one or more food ingredients which are heated to a temperature at or above 85 degrees Fahrenheit. In some embodiments, a compound for treating constipation comprises a mixture of one or more laxatives and one or more food ingredients which are heated to a temperature at or above 80 degrees Fahrenheit. In some embodiments, a compound for treating constipation comprises a mixture of one or more laxatives and one or more food ingredients which are heated to a temperature at or above 75 degrees Fahrenheit.

In some embodiments, the duration of heating is 1 hour or greater. In some embodiments, the duration of heating is 45 minutes or greater. In some embodiments, the duration of heating is 30 minutes or greater. In some embodiments, the duration of heating is 20 minutes or greater. In some embodiments, the duration of heating is 15 minutes or greater. In some embodiments, the duration of heating is 10 minutes or greater. In some embodiments, the duration of heating is 5 minutes or greater. In some embodiments, the duration of heating is 1 minute or greater.

In some embodiments, a compound for treating constipation further comprises a binder, wherein a binder is an element used to bind one or more elements of the compound together. In some embodiments, the binder is edible. In some embodiments, the binder comprises cocoa butter. In some embodiments, the binder comprises coconut oil. In some embodiments, a binder is heated and the elements of the compound are added to the heated binder. In some embodiments, one or more elements of the compound are added to the binder at the hottest temperature to which it is heated. In some embodiments, one or more elements of the compound are added to the heated binder after it has cooled. In some embodiments, the binder is a liquid when heated so that when mixed with the elements of the compound, the elements coalesce or are “bound” together by the binder. In an exemplary embodiment, a binder in the compound is in a solid state at room temperature and a liquid state at a relatively small increase in temperature above room temperature. This exemplary binder is used in binding the elements of the compound when a solid compound is used such as, for example, a bar. In the liquid state the binder can coalesce the elements, and, as stated, the exemplary binder is in a liquid state at temperatures slightly above room temperature, thus the other elements of the compound are not heated due to the binder to a large extent which is especially important with respect to the laxatives that are damaged at high temperatures. In some embodiments, a binder is heated to a temperature at or above 95 degrees Fahrenheit and then one or more elements of the compound for treating constipation are mixed together with it. In some embodiments, a binder is heated to a temperature at or above 90 degrees Fahrenheit and then one or more elements of the compound for treating constipation are mixed together with it. In some embodiments, a binder is heated to a temperature at or above 85 degrees Fahrenheit and then one or more elements of the compound for treating constipation are mixed together with it. In some embodiments, a binder is heated to a temperature at or above 80 degrees Fahrenheit and then one or more elements of the compound for treating constipation are mixed together with it. In some embodiments, a binder is heated to a temperature at or above 75 degrees Fahrenheit and then one or more elements of the compound for treating constipation are mixed together with it. In some embodiments, a binder is heated to a temperature at or above 70 degrees Fahrenheit and then one or more elements of the compound for treating constipation are mixed together with it. In some embodiments, after being heated to an initial temperature, the binder is allowed to cool 30degrees from the initial temperature before one or more elements of the compound for treating constipation are added. In some embodiments, after being heated to an initial temperature, the binder is allowed to cool 25 degrees from the initial temperature before one or more elements of the compound for treating constipation are added. In some embodiments, after being heated to an initial temperature, the binder is allowed to cool 20 degrees from the initial temperature before one or more elements of the compound for treating constipation are added. In some embodiments, after being heated to an initial temperature, the binder is allowed to cool 15 degrees from the initial temperature before one or more elements of the compound for treating constipation are added. In some embodiments, after being heated to an initial temperature, the binder is allowed to cool 10 degrees from the initial temperature before one or more elements of the compound for treating constipation are added. In some embodiments, after being heated to an initial temperature, the binder is allowed to cool 5 degrees from the initial temperature before one or more elements of the compound for treating constipation are added. It is understood that numerous other edible binders are suited, non-limiting examples of which include lard, vegetable shortening, palm oil, butter, or margarine.

In some embodiments, a composition for treating constipation comprises 40% or greater laxative by weight. In some embodiments, a composition for treating constipation comprises 45% or greater laxative by weight. In some embodiments, a composition for treating constipation comprises 50% or greater laxative by weight. In some embodiments, a composition for treating constipation comprises 55% or greater laxative by weight. In some embodiments, a composition for treating constipation comprises 60% or greater laxative by weight. In some embodiments, a composition for treating constipation comprises 65% or greater laxative by weight. In some embodiments, a composition for treating constipation comprises 70% or greater laxative by weight. In some embodiments, a composition for treating constipation comprises 75% or greater laxative by weight. In some embodiments, a composition for treating constipation comprises 80% or greater laxative by weight. In some embodiments, a composition for treating constipation comprises 85% or greater laxative by weight. In some embodiments, a composition for treating constipation comprises 90% or greater laxative by weight. In some embodiments, a composition for treating constipation comprises 95% or greater laxative by weight.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition for treating constipation comprising:
a laxative; and
an anxiolytic.

2. The composition of claim 1, wherein the laxative comprises PEG.

3. The composition of claim 1, wherein the laxative comprises PEG 3350.

4. The composition of claim 1, wherein the anxiolytic is a medication that increases serotonin within a body of an individual.

5. The composition of claim 1, wherein the anxiolytic comprises Buspirone.

6. The composition of claim 1, wherein the anxiolytic comprises a tricyclic anti-depresant.

7. The composition of claim 1, wherein the anxiolytic comprises a selective serotonin reuptake inhibitor.

8. The composition of claim 7, wherein the selective serotonin reuptake inhibitor comprises Fluoxetine (Prozac).

9. The composition of claim 1, wherein the anxiolytic comprises Propranolol.

10. The composition of claim 1, wherein the anxiolytic comprises a serotonin precursor.

11. The composition of claim 8, wherein the serotonin precursor is one of L-tryptophan, L-phenylalanine, and L-tyrosine.

12. The composition of claim 1, comprising one or more food ingredients, wherein the one or more food ingredients are mixed together with the laxative and the anxiolytic forming a solid food item containing the laxative and the anxiolytic.

13. The composition of claim 1, comprising at least 2 g of fiber.

14. The composition of claim 1, comprising a probiotic.

15. The composition of claim 1, comprising a prebiotic.

16. The composition of claim 1, comprising butyrate.

17. The composition of claim 1, comprising an antibiotic.

18. A composition for treating constipation comprising:
a laxative; and
a motility promoting agent.

19. The composition of claim 18, wherein the laxative comprises PEG.

20. The composition of claim 18, wherein the laxative comprises PEG 3350.

21. The composition of claim 18, wherein the laxative comprises a stimulant laxative.

22. The composition of claim 21, wherein the stimulant laxative comprises bisacodyl.

23. The composition of claim 18, wherein the motility promoting agent comprises neostigmine.

24. The composition of claim 18, wherein the motility promoting agent comprises fiber.

25. The composition of claim 18, comprising one or more food ingredients, wherein the one or more food ingredients are mixed together with the laxative and the motility promoting agent forming a solid food item containing the laxative and the motility promoting agent.

26. The composition of claim 18, comprising a probiotic.

27. The composition of claim 18, comprising a prebiotic.

28. The composition of claim 18, comprising butyrate.

29. The composition of claim 18, comprising an antibiotic.

30. A composition for treating constipation comprising:
a laxative; and
one or more of a probiotic and a prebiotic.

31. The composition of claim 30, wherein the laxative comprises PEG.

32. The composition of claim 30, wherein the laxative comprises PEG 3350.

33. The composition of claim 30, wherein the probiotic is on of Bacillus subtilis, Bacillus clausii, Bacillus cereus, Bacillus coagulans and Bacillus licheniformis.

34. The composition of claim 30, wherein the prebiotic comprises beta-glucan.

35. The composition of claim 30, comprising one or more food ingredients, wherein the one or more food ingredients are mixed together with the laxative and the one or more of the probiotic and the prebiotic.

36. The composition of claim 30, comprising at least 2 g of fiber.

37. The composition of claim 30, comprising butyrate.

38. The composition of claim 30, comprising an antibiotic.
